(19) United States
(12) Patent Application Publication
(10) Pub. No.: US 2022/0387793 A1
(43) Pub. Date: Dec. 8, 2022

(54) ELECTRICAL STIMULATION SYNCHRONIZED WITH PATIENT BREATHING

(71) Applicant: MHS Care-Innovation, LLC, Cleveland, OH (US)

(72) Inventor: Gengqing Song, Solon, OH (US)

(21) Appl. No.: 17/832,265

(22) Filed: Jun. 3, 2022

Related U.S. Application Data
(60) Provisional application No. 63/202,299, filed on Jun. 4, 2021.

(51) Int. Cl. A61N 1/36

(52) U.S. Cl. CPC A61N 1/36007; A61N 1/36139; A61N 1/36153; A61N 1/36157; A61N 1/36175

(57) ABSTRACT

A method of providing electrical stimulation to a patient, to treat a disorder from which the patient suffers, includes: detecting respiration of the patient with a sensor; and repeatedly administering electrical stimulations to a target site of the patient. Suitably, the repeated administration of said electrical stimulations is automatically synchronized with the respiration of the patient as detected by the sensor.

### PRIORITY CLAIM AND CROSS-REFERENCE

This application claims the benefit of U.S. Provisional Application No. 63/202,299 filed Jun. 4, 2021, which is incorporated herein by reference in its entirety.

### BACKGROUND

The following relates to the medical arts. It finds particular application in the treatment of functional gastrointestinal disorders (FGIDs) and/or other like conditions in human patients, and some suitable embodiments disclosed herein will be described with reference thereto at times. In particular, some embodiments described herein relate to a method and/or apparatus for treating a FGID or other like condition in a patient via suitable electrical stimulation that is automatically synchronized with the patient's breathing. Alternatively, the subject matter described herein may likewise be applicable to the treatment of other disorders and/or conditions.

FGIDs, for example, such as functional dyspepsia, irritable bowel syndrome and functional heartburn, can affect a significant portion of the general population worldwide, and can result in significant health care costs, impaired health-related quality of life for patients, etc. Other health conditions that may similarly impact patients include, but are not limited to, gastroesophageal reflux disease (GERD), dysphagia, bloating, abdominal pain and discomfort, nausea, vomiting, gastroparesis, burbulence, intestinal pseudo-obstruction, postoperative ileus, fecal or urinary incontinence, constipation, diarrhea, pancreatitis, ulcerative colitis, Crohn's disease, menstrual cramps, spastic and interstitial cystitis and ulcers, obesity, anorexia nervosa, and bulimia nervosa. FGIDs such as functional dyspepsia and irritable bowel syndrome are common disorders seen by gastroenterologists. The diagnosis of FGIDs has been evolving in response to updated research on diagnostic algorithms. FGIDs can be recognized by the coexistence of multiple morphologic and physiological abnormalities including motility disturbance, visceral hypersensitivity, altered mucosal and immune function, altered gut microbiota, and altered central nervous system processing. For example, some significant pathophysiological abnormalities involved in FGIDs are gastrointestinal (GI) motility disturbance, visceral hypersensitivity, altered autonomic function, and abnormal central nervous system processing. Some common therapies for FGIDs focus on diet and/or lifestyle modifications and/or interventions. However, such diet and/or lifestyle modifications/interventions can have limited therapeutic effectiveness and/or be otherwise less than satisfactory. Moreover, pharmacotherapies available to patient's suffering with FGIDs can lack sufficient efficacy and/or can result in various undesirable side effects.

Accordingly, there remains, in general, a desire to have an effective treatment option for patients suffering with FGIDs and/or other like conditions, for example, which is more potent than conventional methods and/or which can be effective in improving motility disturbance and sensory abnormalities of FGIDs as well as in ameliorating symptoms experienced by patients. Hence, there is described herein an inventive method, device and/or system to address the above-identified concerns.

### BRIEF DESCRIPTION

This Brief Description is provided to introduce concepts related to the present specification. It is not intended to identify essential features of the claimed subject matter nor is it intended for use in determining or limiting the scope of the claimed subject matter. The exemplary embodiments described below are not intended to be exhaustive or to limit the claims to the precise forms disclosed in the following Detailed Description. Rather, the embodiments are chosen and described so that others skilled in the art may appreciate and understand the principles and practices of the subject matter presented herein.

In one suitable embodiment, a method of providing electrical stimulation to a patient, to treat a disorder from which the patient suffers, includes: detecting respiration of the patient with a sensor; and repeatedly administering electrical stimulations to a target site of the patient. Suitably, the repeated administration of the electrical stimulations is automatically synchronized with the respiration of the patient as detected by the sensor.

In another suitable embodiment, an apparatus for providing electrical stimulation to a patient, to treat a disorder from which the patient suffers, includes: a sensor which is to be fitted to the patient, the sensor detecting respiration of the patient to which it is fitted; and an electric power supply, the electric power supply being controlled to provide electrical signals to an electrode such that repeated electrical stimulations are automatically administered to a target site of the patient via the electrode in synchronization with the respiration of the patient detected by the sensor.

In still another suitable embodiment, a medical treatment device that provides electrical stimulation to a user includes: a sensor that monitors a respiration cycle of the user; an electrode that administers electrical stimulations to a target site of the user, wherein the target site is one of a nerve of the user and an organ of the user and the electrode comprises one of a transcutaneous pad positioned on a skin of the user, a percutaneous needle inserted through the skin of the user, and a lead implanted within the user; and an electric power supply electrically coupled to the electrode, the electric power supply being controlled to provide electrical signals to the electrode such that repeated electrical stimulations are administered to the target site of the user via the electrode in automatic synchronization with the respiration cycle of the user as monitored by the sensor.

Numerous advantages and benefits of the subject matter disclosed herein will become apparent to those of ordinary skill in the art upon reading and understanding the present specification. It is to be understood, however, that the detailed description of the various embodiments and specific examples, while indicating preferred and/or other embodiments, are given by way of illustration and not limitation.

### BRIEF DESCRIPTION OF THE DRAWINGS

The following Detailed Description makes reference to the figures in the accompanying drawings. However, the inventive subject matter disclosed herein may take form in various components and arrangements of components, and in various steps and arrangements of steps. The drawings are only for purposes of illustrating some exemplary and/or preferred embodiments and are not to be construed as limiting. Further, it is to be appreciated that the drawings may not be to scale.

FIG. 1 diagrammatically illustrates a medical treatment device and/or apparatus in accordance with some embodiments disclosed herein.

FIG. 2 shows a graph on which is plotted an exemplary respiration signal provided by a respiration sensor in accordance with some embodiments disclosed herein. The y-axis is unitless and shows the respiration signal magnitude. The x-axis is time.

FIG. 3 is an illustration showing a stimulation signal automatically synchronized with the respiration cycle of a user, and in particular, the start of inhalation and/or inspiration of the user.

FIG. 4 is a flow chart showing a method of administering electrical stimulation to a patient in accordance with some embodiments disclosed herein.

FIG. 5 is a diagram showing an example of a monitoring system, in accordance with some embodiments of the present disclosure.

### DETAILED DESCRIPTION

For clarity and simplicity, the present specification shall refer to structural and/or functional elements, relevant standards, algorithms and/or protocols, and other components, methods and/or processes that are commonly known in the art without further detailed explanation as to their configuration or operation except to the extent they have been modified or altered in accordance with and/or to accommodate the preferred and/or other embodiment(s) presented herein. Moreover, the apparatuses and methods disclosed in the present specification are described in detail by way of examples and with reference to the figures. Unless otherwise specified, like numbers in the figures indicate references to the same, similar or corresponding elements throughout the figures. It will be appreciated that modifications to disclosed and described examples, arrangements, configurations, components, elements, apparatuses, methods, materials, etc. can be made and may be desired for a specific application. In this disclosure, any identification of specific materials, techniques, arrangements, etc. are either related to a specific example presented or are merely a general description of such a material, technique, arrangement, etc. Identifications of specific details or examples are not intended to be, and should not be, construed as mandatory or limiting unless specifically designated as such. Selected examples of apparatuses and methods are hereinafter disclosed and described in detail with reference made to the figures. Indeed, the following disclosure provides many different embodiments, or examples, for implementing different features of the provided subject matter. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

Further, spatially relative terms, such as “left,” “right,” “side,” “back,” “rear,” “behind,” “front,” “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

In general, the present specification describes a method, system, device and/or apparatus that provides automatically synchronized electrical stimulation to a user or patient to treat a disorder, ailment and/or condition from which the user or patient may be suffering. More specifically, the disclosed method, system, device and/or apparatus automatically delivers or otherwise provides an intermittent or periodic or other pattern of electrical stimulation to a user or patient that is synchronized with the user/patient's breathing or respiration. In some suitable embodiments, the system, device and/or apparatus may include one or more breath or respiration sensors that detect the breathing and/or respiration of a user/patient; one or more stimulation controllers or the like, e.g., employing a suitable algorithm or the like to identify when (i.e., at a given point in time) to deliver the electrical stimulation such that the electrical stimulation is automatically synchronized with the breathing/respiration of the user/patient; one or more electrical regulators and/or controllers to control and/or manage parameters of the electrical stimulation provided, e.g., such as the voltage, current, frequency, pulse width, duration, waveform and/or other suitable parameters of the provided electrical stimulation; one or more electrical power supplies to produce an electrical signal, pulse or the like in accordance with the parameters set or otherwise established by the electrical regulator(s)/controller(s); and one or more electrodes operatively connected to the electrical generator(s) to deliver and/or administer electrical stimulation to one or more target sites.

In this regard, respiration involves cycles of inhalation and exhalation. Inhalation is also known as inspiration and is the phase in which air enters the lungs. Exhalation is also known as expiration and is the phase in which air exits the lungs.

In some suitable embodiments, monitoring and/or feedback features may also be provided. Such an additional feature may be implemented via or with the aid of one or more smart devices or computers or the like that utilize or support a one or more suitable applications running thereon and/or executed thereby. In practice, these applications can be designed and/or provisioned to perform various functions, including collecting symptom and/or laboratory data; performing sensor-assisted pathophysiological measurements; and/or providing biofeedback functions. In some embodiments, suitable smart devices may include of any number of portable electronic devices, e.g., including but not limited to smartphones, handheld tablets, laptop computers, desktop computers, and any other similar electronic device or computer. In practice, the monitoring and/or feedback features may employ collected or otherwise acquired data to evaluate the effectiveness of previously provided electrical stimulation and return feedback utilized by the stimulation controller and/or electrical regulator/controller to modify the synchronization, timing, pattern, parameters and/or form of future provided electrical stimulation.

In general, an advantage of some embodiments disclosed herein is the potential to be more potent than traditional electrical stimulation alone, e.g., without synchronization to a user/patient's breathing/respiration. More specifically, this more potent stimulation has the potential to improve major pathogenesis for FGIDs, e.g., such as gut motor and sensory abnormalities; thereby decreasing symptom severity and improving the quality of life in FGID patients. Some suitable embodiments herein can be applicable to a variety of disease indications including, but not limited to, FGIDs, gastroesophageal reflux disease (GERD), postoperative ileus, abdominal pain and discomfort, pancreatitis, ulcerative colitis, Crohn's disease, menstrual cramps, spastic and interstitial cystitis and ulcers, obesity, anorexia nervosa, and bulimia nervosa. Some embodiments described herein provide therapeutic treatment for and/or alleviate symptoms of various conditions and/or disorders, e.g., including, but not limited to: FGIDs, functional dyspepsia, irritable bowel syndrome, gastroesophageal reflux disease (GERD), heartburn, bloating, postoperative ileus, abdominal pain and discomfort, early satiety, epigastric pain, nausea, vomiting, burbulence, regurgitation, intestinal pseudo-obstruction, anal incontinence, gastroesophageal reflux disease, chronic constipation, gastroparesis, pancreatitis, ulcerative colitis, Crohn's disease, menstrual cramps, spastic and interstitial cystitis and ulcers, obesity, anorexia nervosa, and bulimia nervosa.

In some suitable embodiments, the electrical stimulation can be administered in a variety of manners to a variety of target sites, including, but not limited to any of the following: electrical stimulation of a nerve (e.g., such as vagal nerve, spinal cord, peripheral nerve, nerve plexus, etc.); electrical stimulation of an organ (e.g., such as segments of gut, liver, pancreas, bladder, etc.); a combination of nerve and organ stimulation. In some suitable embodiments, delivery and/or administration of the electrical stimulation can be via a needle-less transcutaneous route (e.g., using noninvasive adhesive skin electrode pads), a percutaneous route (e.g., using needle electrodes), and/or an implantable route (e.g., using patient implanted electrodes at and/or around the target site of a nerve or an organ). Suitably, any single manner and/or route and/or various combinations thereof can be used to achieve the desired stimulation. Suitably, other stimulation manners and/or routes not mentioned here can also be used to obtain the desired electrical stimulation.

In practice, a variety of different configurations of stimulation electrodes, stimulation target sites and/or stimulation parameters may be employed to treat the patient using one or more electrodes sequentially or otherwise attached to one or more electrical pulse or function generators or the like. For example, one or more electrodes can be used, including but not limited to transcutaneous electrode pads, percutaneous needle electrodes, or implantable leads, for the electrical stimulation of one or more target sites, including but not limited to nerves or organs. In some suitable embodiments, the desired electrical stimulation of selected target sites is achieved by selecting individual electrodes and setting and/or adjusting various stimulation parameters therefor. Examples of stimulation parameters include but are not limited to the shape of the stimulation waveform, amplitude of current or voltage, pulse width, frequency, and anodic or cathodic stimulation, in addition to a number of other controllable parameters.

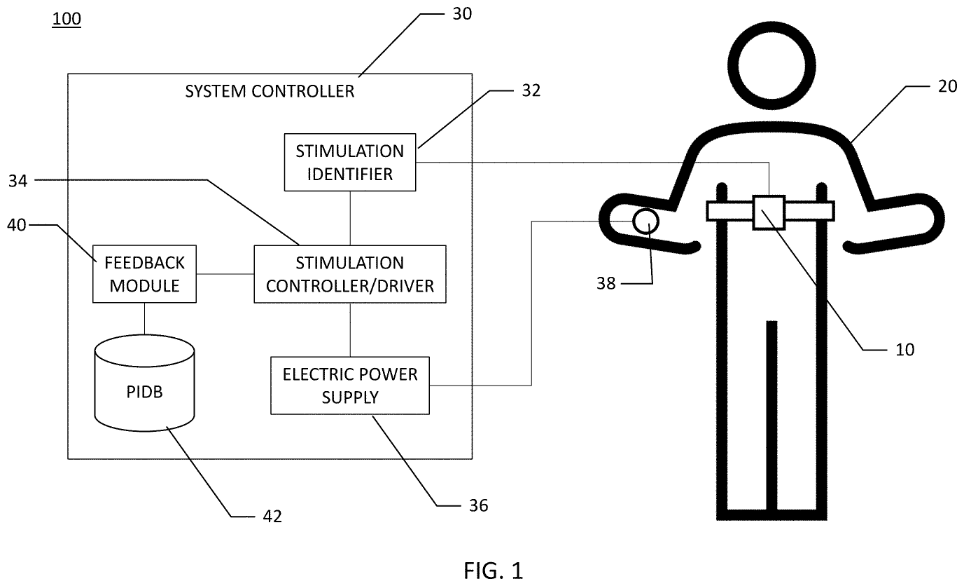

With reference now to FIG. 1, a medical treatment device and/or apparatus 100 includes a respiration sensor 10 that is fitted to and/or worn by a patient or user 20 and measures, detects and/or monitors the breathing and/or respiration of the patient/user 20.

In some suitable embodiments, the sensor 10 may include a flexible strap or other belt or the like that is (i) fitted and/or worn around an upper aspect or region of the trunk, torso or chest of the patient/user 20, e.g., to measure, detect and/or monitor thoracic respiration (i.e., chest breathing), or (ii) fitted and/or worn around a lower aspect or region of the trunk, torso or abdomen of the patient/user 20, e.g., to measure, detect and/or monitor diaphragmatic respiration (i.e., stomach breathing). In general, breathing and/or respiration is generally accompanied by an associated cycle of expansion and contraction of the chest and/or abdomen of an individual. Suitably, the sensor 10 measures, detects and/or monitors respiration and generates a corresponding signal in response to the expansion and contraction of the patient/user's anatomy in the region or area where the sensor is fitted and/or worn. Put another way, during inhalation or inspiration, the patient/user's trunk or torso generally expands thereby applying and/or gradually increasing a force or pressure to the sensor 10. During exhalation or expiration, the patient/user's trunk or torso generally contracts thereby relaxing and/or gradually reducing the force or pressure applied to the sensor 10. Suitably, the sensor 10 generates a voltage or other electrical signal which is proportional to and/or otherwise changes in response to the pressure or force experienced by the sensor 10.

In some suitable embodiments, the sensor 10 may extract force information (f) during the expansion and/or contraction of the patient/user's anatomy during breathing or respiration. For example, the sensor 10 can be a flexible force sensitive resistor (FSR) that produces a voltage change correlating to the pressure applied against the sensor 10 during an anatomy expansion associated with an inhalation breath. In some embodiments, the sensor 10 may employ an electromechanical film, e.g., having a thin porous polypropylene structure, to form a capacitive pressure sensor which produces a voltage change proportional to the anatomical movement associated with respiration (i.e., torso expansion and contraction). In some alternative embodiments, the sensor 10 may comprise a mask or the like worn and/or fitted about the mouth and/or nose of the patient/user 20 in order to sense the airflow therethrough associated with respiration. In some other embodiments, the sensor 10 may comprise an accelerometer positioned on the anatomy of the patient/user 20 in order to sense anatomical movement associated with respiration. In other embodiments, the sensor 10 may comprise a piezoelectric device that produces a signal in response to the pressure and/or force applied thereto. In general, the sensor 10 may be any suitable respiration sensor or combination of one or more suitable sensors that measures, detects and/or monitors breathing and/or respiration of the patient/user 20 and outputs a signal in response thereto and/or based thereon.

In practice, a signal from the respiration sensor 10 is suitably provided as input to a system controller 30 that regulates and/or controls various operations and/or functions of the device or apparatus 100 so as to administer repeated electrical stimulations to a target site of the patient/user 20 which are automatically and/or otherwise synchronized with the respiration of the patient/user 20 as measured, detected and/or monitored by the sensor 10, e.g., in real or near real time. In some suitable embodiments, a signal obtained from the respiration sensor 10 can be transmitted to the system controller 30 via an Analog-to-Digital Converter (ADC).

As shown in FIG. 1, the system controller 30 includes a stimulation identifier 32. Suitably, the stimulation identifier 32 analyzes and/or evaluates the input signal from the sensor 10 to identify a characteristic trait and/or pattern therein which represents a particular point-of-interest (POI) in the patient/user's respiration cycle, such that the administration of the electrical stimulation can be synchronized and/or otherwise timed with reference to that particular POI in the respiration cycle. For example, the stimulation identifier 32 may determine where the starting point of inhalation is and generate a command or signal indicating that starting point, which command or signal is then in turn forwarded or otherwise communicated to a stimulation controller or driver 34 which may be triggered thereby to control and/or regulate an electric power supply 36 to administer electrical stimulation via an electrode 38 to a target site of the patient/user 20 in accordance with one or more stimulation parameters. For example, the stimulation parameters may include, but are not limited to, a shape of a waveform for the electrical stimulation, an amplitude of a current for the electrical stimulation, an amplitude of a voltage for the electrical stimulation, a frequency of the electrical stimulation, a pulse width of the electrical stimulation and a duration of the electrical stimulation.

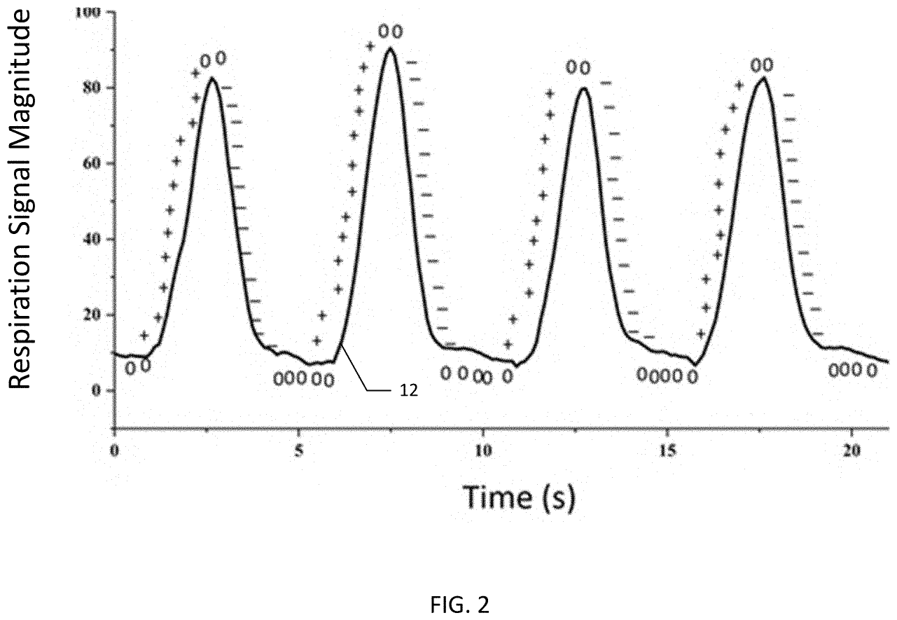

With reference now to FIG. 2, an exemplary respiration signal 12 produced by the sensor 10 is shown plotted on a graph, where the vertical axis of the graph represents a magnitude of the signal 12 in arbitrary units and the horizontal axis of the graph represents time in seconds. As shown in FIG. 2, at various points along the plot, a slope of the signal 12 is positive, indicated in FIG. 2 by plus signs “+”. For example, where the slope of the signal 12 is generally positive may correspond to inhalation by the patient/user 20 as detected by the sensor 10. At various other points along the plot, a slope of the signal 12 is negative, indicated in FIG. 2 by minus signs “−”. For example, where the slope of the signal 12 is generally negative may correspond to exhalation by the patient/user 20 as detected by the sensor 10. At yet other various points along the plot, a slope of the signal 12 is zero, essentially zero, substantially flat or significantly negligible in magnitude, indicated in FIG. 2 by a zero “0”. For example, these points may correspond to transitions and/or pauses between exhalation and inhalation and vice versa by the patient/user 20 as detected by the sensor 10.

In some suitable embodiments, the stimulation identifier 32 may analyze and/or evaluate the respiration signal 12 input to the system controller 30 to determine the slope thereof and identify a characteristic trait and/or pattern in the slope which represents a particular point of interest in the patient/user's respiration. For example, the stimulation identifier 32 may determine the start of inhalation by recognizing one or more points where the slope of the signal 12 is essentially zero, substantially flat and/or negligible in magnitude, followed by recognizing a positive slope developing in the signal 12. When such a pattern in the slope of the respiration signal 12 is recognized, the stimulation identifier 32 may communicate to the stimulation controller or driver 34 that inhalation has started. More generally, after detecting and extracting the breath or respiration information from the sensor 10, a respiration parameter vector can be created, e.g., representing the slope or other suitable characteristic of the respiration signal 12. This vector parameter can be updated regularly and communicated to the stimulation identifier 32 which recognizes and/or identifies a characteristic trait and/or pattern in the respiration parameter representing a particular POI in the patient/user's respiration. Accordingly, the stimulation controller or driver 34 may be triggered by a signal or command or other communication received from the stimulation identifier 32 such that the stimulation controller/driver 34 controls and/or regulates the electric power supply 36 to administer electrical stimulation via the electrode 38 to a target site of the patient/user 20 in automatic synchronization with and/or relatively timed with respect to the POI identified by the stimulation identifier 32.

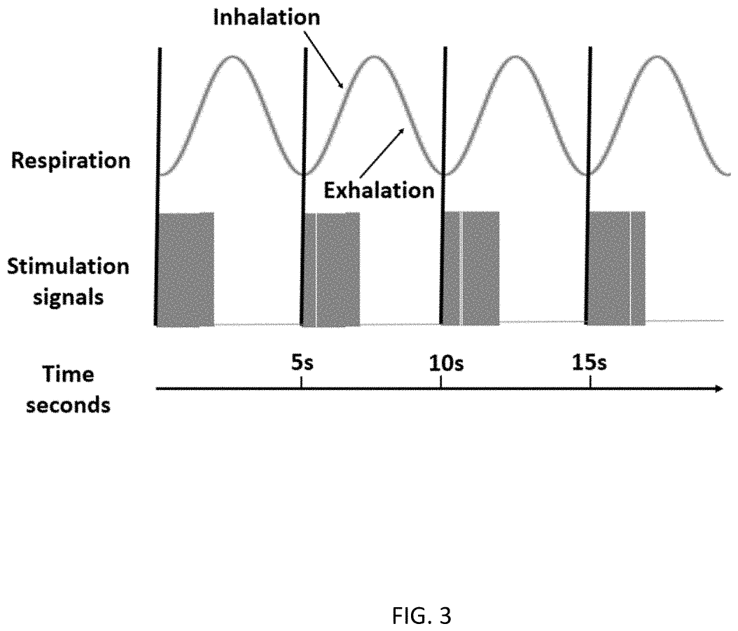

The automatic synchronization of a stimulation signal with detected patient respiration is more specifically illustrated by way of a non-limiting example shown in FIG. 3. In this particular non-limiting example, the stimulation is automatically synchronized with a start of inhalation/inspiration as detected by the sensor. As shown in FIG. 3, the detected respiration cycle or signal (e.g., produced by the sensor 10) is represented by a sinusoidal waveform, with the start of inhalation or inspiration occurring as the slope of the respiration waveform begins going positive. Exhalation or expiration follows, with the slope of the respiration waveform going negative. The stimulation signal is illustrated here as a square waveform, with the stimulation being applied at the starting point of inspiration. The stimulation is applied for roughly one half of the inspiration portion of the respiration signal. As shown, the system can accurately determine the starting point of the inhalation breath and provide electrical stimulation that correlates with the breath, providing a stimulation pattern that correlates to the breathing pattern of the user.

In practice, the electrode 38 may comprise one or more of: a noninvasive electrode pad which is adhered or otherwise positioned on the skin of the patient/user 20 to deliver and/or administer electrical stimulation (e.g., acustimulation) to the target site via a needle-less transcutaneous route; a needle electrode which is inserted through the skin of the patient/user 20 to deliver and/or administer electrical stimulation (e.g., electro-acupuncture) to the target site via a percutaneous route; and/or a lead surgically or otherwise inserted in the patient/user 20 to deliver and/or administer electrical stimulation to the target site via an implantable route. Suitably, the target site may include a nerve or an organ of the patient/user 20 and the electrode 38 may be positioned and/or located in, at, near, or on the same or otherwise in therapeutic proximity thereto. Suitably, the electrode 38 may be anodic or cathodic. As shown in FIG. 1, the electrode 38 is suitably coupled to and/or in electrical communication with the power supply 36 that selectively energizes and/or provides an electrical signal thereto under the direction of the stimulation controller/driver 34.

In some suitable embodiments, the stimulation controller/drive 34 is triggered by and/or otherwise responsive to a signal and/or command or other like communication received from the stimulation identifier 32 to control and/or regulate the electric power supply 36 such that the electric power supply 36 energizes or otherwise provides an electrical signal to the electrode 38 which is operatively coupled to and/or in electrical communication with the power supply 36, thereby providing electrical stimulation to the target site via the electrode 38 in automatic synchronization with and/or timed relative to the patient/user's respiration as detected by the sensor 20. In practice, the power supply 36 may be an electric pulse or function generator that supplies an electrical signal to and/or otherwise energizes the electrode 38 in accordance with determined, selected, set and/or otherwise established stimulation parameters and/or under the control and/or regulation of the stimulation controller/driver 34. As previously mentioned herein, the stimulation parameters may include for example, but are not limited to, a shape of a waveform for the electrical stimulation, an amplitude of a current for the electrical stimulation, an amplitude of a voltage for the electrical stimulation, a frequency of the electrical stimulation, a pulse width of the electrical stimulation and a duration of the electrical stimulation. Suitably, the stimulation controller/driver 34 may determine, select, set and/or otherwise establish the stimulation parameters and control or regulate the power supply 36 accordingly.

As shown in FIG. 1, the system controller 30 may further include a feedback module 40. In some suitable embodiments, the feedback modules 40 may collect, receive and/or otherwise obtain patient data from one or more appropriate sources. For example, suitable sources for patient data may include, but are not limited to: one or more sensors that measure, detect and/or otherwise monitor biophysiological data or the like; manually entered or other like inputs from the patient/user 20, a medical professional or technician, or other individual, e.g., related to symptoms being experienced and/or changes therein, eating habits, meals, sleep quality, patterns and/or habits, and other lifestyle data of the patient/user 20, laboratory test data, test data obtained from biological samples, etc. Suitably, the patient data may be recorded, updated and/or maintained in a patient information database (PIDB) 42. The PIDB 42 may further maintain timestamped or otherwise identified historical records of the patient data along with the program and/or particulars of any electrical stimulation being administered during various periods of time. For example, historical patient data may be stored and tracked relative to date and/or time, such as by monthly, weekly, daily, hourly and/or by a custom date and/or time range, along with the particulars of any electrical stimulation administered during the respective time-period. Suitably, the particulars of any electrical stimulation administered, including the stimulation parameters employed in accordance with various instances, designation of target sites, etc., may be noted by the feedback module 40 and/or received from the system controller 30 and/or the stimulation controller/driver 34 at the time of administration.

In some suitable embodiments, the feedback module 40 may employ artificial intelligence, advanced machine learning, suitable algorithms and/or the like to review and/or analyze the patient data, e.g., from the PIDB 42, to evaluate the historic effectiveness of particular electrical stimulations previously administered during a given period of time, and based thereon select, adjust, recommend and/or establish new stimulation parameters to be employed in connection with some future administration of electrical stimulations. In practice, an output of the feedback module 40 may be communicated to the stimulation controller/driver 34 such that the stimulation parameters employed by the stimulation controller/driver 34 are set, tuned and/or adjusted in accordance with the directive of the feedback module 40.

In some suitable embodiments, the feedback module 40 may be separate from the system controller 30 and wirelessly or otherwise in communication therewith. For example, the feedback module 40 may be implemented via a suitable application or the like running and/or executed on a suitable smart device, smartphone, laptop or computer.

In general, the feedback module 40 acts as a closed-loop or other like feedback mechanism for the device or apparatus 100, e.g., so that the stimulation parameters may be automatically or otherwise tuned or adjusted for future administrations of electrical stimulations based on a recognized or detected effectiveness of prior administered electrical stimulations. In practice, the feedback module 40 may collect patient data, e.g., such as patient symptoms, blood, stool, and/or other pathophysiological measurements from the PIDB 42; and therefrom may calculate a score upon which a biofeedback function may be based (e.g., in accordance with changes to the score), which represents an effectiveness and/or response to the treatment being administered at the time. For example, if the score is increasing this may represent that symptoms are getting worse over time, indicating a less than suitable response to the stimulation, then the feedback module 40 will communicate with the system controller 30 and/or stimulation controller/driver 34 such that another set of stimulation parameters is selected from a pool of stimulation regimens. Conversely, if the score is decreasing this may represent that symptoms are abating, suggesting positive effects of the stimulation, then the feedback module 40 will not communicate any change in the stimulation parameters.

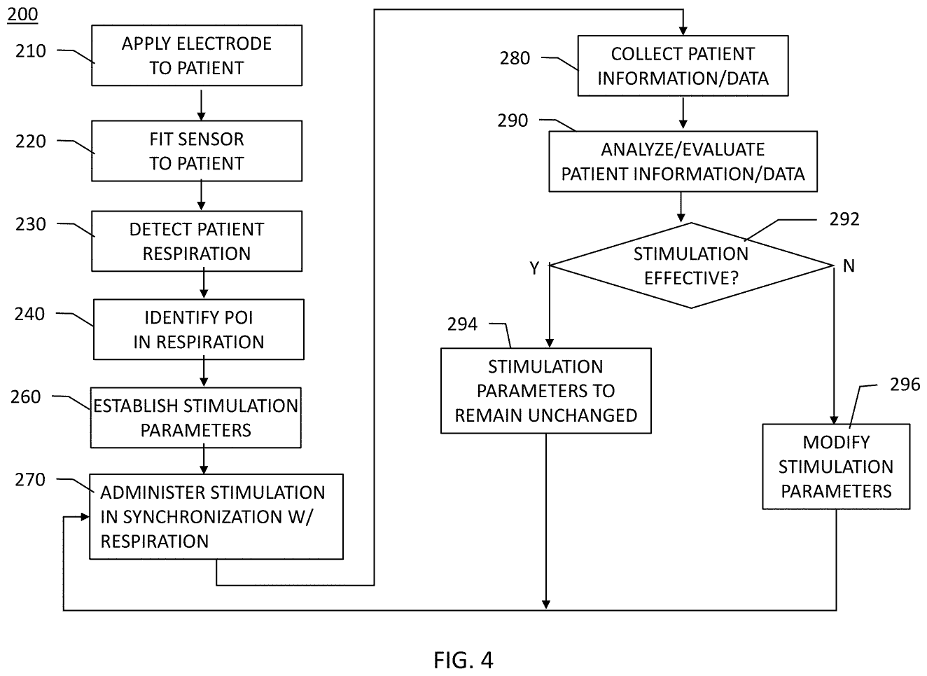

With reference now to FIG. 4, a method 200 in accordance with some suitable embodiments is disclosed by way of illustration in the depicted flowchart.

At step 210, an electrode (e.g., such as the electrode 38) is applied to a patient (e.g., such as the patient/user 20). For example, the electrode may be a pad which is adhered, stuck or otherwise positioned on the patient's skin, a needle which is inserted through the patient's skin, or a lead which is surgically or otherwise implanted within the patient. In practice, the electrode may be applied on, in, suitably near or otherwise therapeutically proximate to a desired or selected target site (e.g., such as a nerve or organ) of the patient where electrical stimulations are to be administered.

At step 220, a breathing or respiration sensor (e.g., such as the sensor 10) is fitted to the patient. While steps 210 and 220 are shown in series one after the other in the illustrated flow chart, it is to be appreciated that in practice the steps 210 and 220 may be performed in any order and/or in parallel.

At step 230, the sensor fitted to the patient in step 220 measures, detects and/or monitors the patient's breathing and/or respiration. In practice, the sensor may output a signal in response to and/or based upon the measured, detected and/or monitored respiration of the patient.

At step 240, a POI within the patient's breathing or respiration is recognized and/or identified from the respiration measured, detected and/or monitored by the sensor fitted to the patient in step 220, i.e., the POI is recognized and/or identified based on the signal output from the sensor fitted to the patient in step 220. Suitably, the POI recognized and/or identified in step 240 is a periodic, intermittent or otherwise reoccurring point within the patient's respiration cycle, e.g., such as the start of inhalation.

At step 260, a set of stimulation parameters are set, determined, adjusted and/or otherwise established. For example, the set of stimulation parameters may include, but is not limited to, any one or more of the following: a shape of a waveform to be used for electrical stimulations, an amplitude of a current to be used for electrical stimulations, an amplitude of a voltage to be used for electrical stimulations, a frequency to be used for electrical stimulations, a pulse width to be used for electrical stimulations and a duration to be used for electrical stimulations. Initially, the stimulation parameters may be set to baseline or nominal values or levels or otherwise selected as desired, for example, by the patient, a medical professional, technician or other individual, etc. In some embodiments, an initial baseline or nominal values or levels can be preprogrammed or provisioned, e.g., such as in the system controller 30 and/or stimulation controller/driver 34.

At step 270, in synchronization with the patient's respiration and/or timed relative to the POI recognized and/or identified in step 240, the electrode applied to the patient in step 210 is repeatedly energized and/or provided an electrical signal (e.g., by the electric power supply 36) in accordance with the stimulation parameters established in step 260, thereby repeatedly administering electrical stimulations to the target site of the patient in automatic synchronization with the patient's respiration and/or timed relative to the POI recognized and/or identified in step 240. For example, in real time or near real time while the patient's respiration cycle is sensed by the sensor fitted to the patient in step 220, when the POI is recognized and/or identified in the patient's respiration cycle at step 240, an electrical stimulation, timed with respect and/or relative to the POI, is administer to the target site of the patient by energizing or supplying an electrical signal to the electrode applied to the patient in step 210, using the stimulation parameters established in step 260.

At step 280, patient information and/or data is collected. For example, the patient information and/or data may include historical records of the patient's symptom severity, biological, blood, stool, laboratory and/or other test results, etc. for a given time-period, correlated with information and/or data regarding prior administrations of electrical stimulations provided to the patient over that time-period. Suitably, the patient information and/or data may be maintained in the PIDB 42 and includes the stimulations parameters used for prior administrations of the electrical stimulations provided to the patient.

At step 290, the patient information and/or data collected in step 280 is analyzed and/or evaluated to determine an effectiveness of electrical stimulations administered previously over a given time-period, e.g., the effectiveness at treating the patient's condition (e.g., such as FGIDs) and/or reducing symptom severity. At decision step 292, if it is determined in step 290 that the prior administered electrical stimulations had been sufficiently effective, then the method 200 branches to step 294, otherwise if it is determined in step 290 that the prior administered electrical stimulations had not been sufficiently effect, then the method branches to step 296.

At step 294, it is indicated that the stimulation parameters should remain unchanged (i.e., because the previously used stimulation parameters were resulting in sufficiently effective treatment and/or symptom alleviation), and the method 200 in turn loops back to step 260 where the set of stimulation parameters is again established accordingly, i.e., without changes as indicated in step 294. Otherwise, at step 296, it is indicated that one or more of the stimulation parameters is to be altered, adjusted or modified (i.e., because the previously used stimulation parameters were not resulting in sufficiently effective treatment and/or symptom alleviation), and the method 200 in turn loops back to step 260 where the set of stimulation parameters is again established accordingly, i.e., with one or more of the stimulation parameters being altered, adjusted and/or modified as indicated in step 296.

Referring back to FIG. 1, a single electrode 38 and power supply 36 have been shown for simplicity and/or clarity herein. It is to be appreciated nonetheless that in practice one or more similar electrodes and/or one or more similar power supplies electrically coupled thereto may be likewise employed to administer electrical stimulations to one or more target sites in automatic synchronization with the respiration of the patient/user 20 as detected by the respiration sensor 10. Additionally, while a single respiration sensor 10 has been illustrated in FIG. 1 for simplicity and/or clarity herein, it is also to be appreciated that in practice a plurality of like respiration sensors 10 may be likewise employed to measure, detect and/or monitor the respiration of the patient/user 20, and signals from such multiple sensors may be combined or otherwise employed to establish or calculate a respiration model representative of the patient/user's respiration (e.g., which model may take a form similar to the respiration signal 12 shown in FIG. 2).

As described herein various elements and/or components (e.g., such as the sensor 10, the system controller 30, the stimulation identifier 32, the stimulation controller/driver 34, the power supply 36, the electrode 38, the feedback module 40 and PIDB 42) exchanged, transmit and/or receive various signals, commands, data, messages, information and the like. In practice, such exchanges, transmissions and/or receptions may be carried out over suitable wired or wireless connections established between the respective elements and/or components.

In some suitable embodiments, one or more user interfaces (UIs) may be provided, e.g., in connection with device or apparatus 100. The UIs may include one or more input and/or output devices that allow the patient/user 20, a medical professional or other individual to suitably interact with the device or apparatus 100. For example, a UI may be provided on a smartphone or other mobile device or a laptop or other computer that is in operative communication with the device or apparatus 100, e.g., in wired or wireless communication with the system controller 30. In some embodiments, the UI may selectively display and/or output a graphical representation of the patient/user's respiration as detected by the sensor 10, e.g., in a graph such as the one depicted in FIG. 2. In some suitable embodiments, the UI may selectively display and/or output a graphical representation of the electrical stimulations administered, e.g., shown as a waveform superimposed or otherwise registered with the graphical representation of the patient/user's respiration. The UI may also allow manual entry and/or other input of patient information and/or data maintained in the PIDB 42. The UI may also allow output and/or review of patient information and/or data from the PIDB 42. In some suitable embodiments, the stimulation parameters may be manually entered and/or otherwise input via the UI interface, e.g., either as initial nominal values or levels or to otherwise manually select the same as desired. In some suitable embodiments, currently prescribed or otherwise established stimulation parameters may be displayed and/or otherwise output via the UI for review by the patient/user 20 or a medial professional or other individual.

In some suitable embodiments, the UI and/or a corresponding application, e.g., running the patient/user's smartphone or other like mobile device, allows the patient/user 20 to track their daily symptoms, sleep quality, and record daily meals. The UI may selectively include a survey interface that is used to input daily symptoms and sleep quality. In some embodiments, daily meals can be input and/or entered via the UI as photographs taken with the patent/user's mobile device or otherwise. Suitably, the mobile device, the UI and/or application can automatically assign and record a current date and timestamp to the meal entry so that any correlations of symptoms can be traced back to a particular meal. In some suitable embodiments, the UI may provide an interactive icon or button or link which can be selected by the patient/user 20 to input the time that they go to sleep. Suitably, all data and/or information entered via the UI, mobile device and/or application can be communicated to and/or maintained in the PIDB 42. In some suitable embodiments, the PIDB 42 is a Health Insurance Portability and Accountability Act of 1996 (HIPAA) compliant medical records database, which can be remotely stored and/or accessed securely via a suitable data communications network, e.g., such as the Internet or the like.

In some embodiments, a sensor-assisted customized monitoring system is provided, for example, for users with GERD. Suitably, the UI may display or otherwise output different functions and/or options that are noted with tabs to allow the ability to visually display summary data based on the analysis of historical user input data to track changes in symptoms over time, the quality and length of sleep, and daily diet and nutrition information. In some embodiments, an analyzer can be embedded into the system and/or otherwise employed that can provide general analysis of the data and determine any correlations between sleep, symptoms, and diet through a visualization on the UI. In some suitable embodiments, the UI may allow a patient/user 20 to input daily symptoms through a data collection mechanism, e.g., such as a survey. The survey may record with a date/time stamp that can be used to refer back to the data chronologically in the future and look at any emerging patterns over time. In some embodiments, another feature of the UI may allow the user to answer a few questions that can be used to determine the sleep quality assessment. In yet further embodiments, another feature of the UI may allow the user to record daily meals and nutrition information through the use of the mobile device's camera to upload a photograph of their meals. Optionally, one or more machine learning algorithms or the like is utilized to detect the type of food and estimate the quantity of food through image processing and data extraction. Another optional feature of the UI and/or application will allow the patient/user 20 to input their sleep time by selecting a “go to bed” option which will then determine and record the user's sleep time. Suitably, the various information and/or data collected via the UI may be maintained in the PIDB 42 and can be provided to a healthcare provider at a later time to allow correlation and determination of symptom diagnosis and potential treatment options.

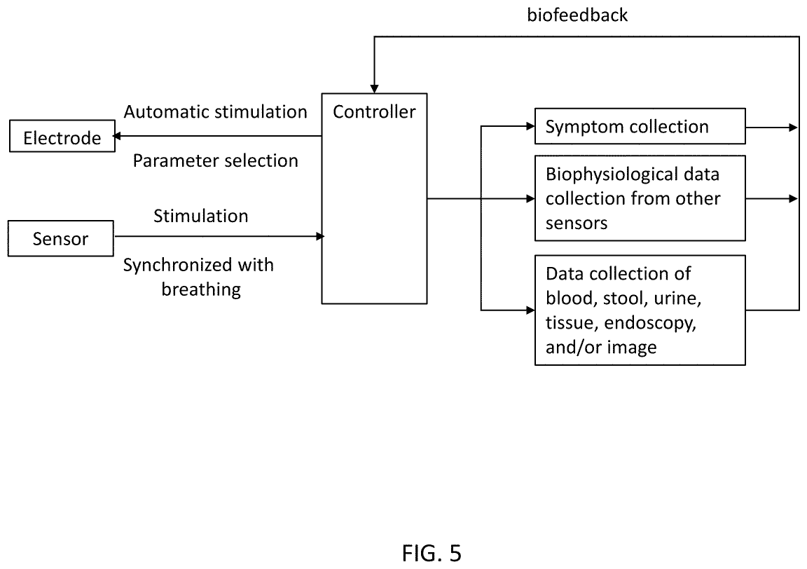

FIG. 5 is an example illustration of a monitoring system. The controller sends stimulation signals to the electrode and receives signals from the sensor, such that the stimulation can be synchronized with the breathing of the patient (particularly the start of inspiration). The controller can also collect symptom, biophysiological data from other sensors, and laboratory data for example from other measurements such as from blood, stool, urine, tissue, endoscopy, other images, and the like. Biofeedback can be provided to the controller based on this other data.

Without limitation, the following are some examples of suitable therapies and/or treatment options that may be provided, for example, with the medical treatment device and/or apparatus shown in FIG. 1 and/or the method shown in FIG. 4:
Vagal nerve (or vagal nerve branch(es)) electrical stimulation in automatic synchronization with breathing can be delivered through transcutaneous, percutaneous or implantable routes/electrodes for treatment of inflammation bowel disease, GI motility disorders, chronic abdominal pain, epilepsy, depression, stroke rehabilitation, or other diseases; for example, including implantable vagal nerve stimulation in automatic synchronization with breathing, percutaneous auricular vagal nerve stimulation in automatic synchronization with breathing, and/or transcutaneous cervical vagal nerve stimulation in automatic synchronization with breathing;
Spinal cord electrical stimulation in automatic synchronization with breathing can be delivered through implantable, percutaneous, or transcutaneous routes/electrodes for treatment of chronic pain, GI motility disorders, or other diseases; for example, including implantable spinal cord electrical stimulation in automatic synchronization with breathing, percutaneous spinal cord electrical stimulation in automatic synchronization with breathing, and/or transcutaneous spinal cord electrical stimulation in automatic synchronization with breathing;
Sacral nerve electrical stimulation in automatic synchronization with breathing can be delivered through implantable, percutaneous, or transcutaneous routes/electrodes for treatment of fecal incontinence, inflammatory bowel disease, GI motility disorders, or other diseases; for example, including implantable sacral nerve electrical stimulation in automatic synchronization with breathing, percutaneous sacral nerve electrical stimulation in automatic synchronization with breathing, and/or transcutaneous sacral nerve electrical stimulation in automatic synchronization with breathing;
Peripheral nerve electrical stimulation, for example, such as tibial nerve electrical stimulation, in automatic synchronization with breathing can be delivered through implantable, percutaneous, or transcutaneous routes/electrodes for treatment of urinary incontinence, GI motility disorders, or other diseases; for example, including percutaneous tibial nerve electrical stimulation in automatic synchronization with breathing and/or transcutaneous tibial nerve electrical stimulation in automatic synchronization with breathing;
Organ (for example, such as gut, bladder, pancreas, liver, or kidney) electrical stimulation in automatic synchronization with breathing can be delivered through implantable, percutaneous, or transcutaneous routes/electrodes for treating GI disease, esophageal diseases, diabetes, obesity or other diseases; for example, including implantable gastric electrical stimulation in automatic synchronization with breathing and/or percutaneous gastric electrical stimulation in automatic synchronization with breathing;
Implantable deep brain electrical stimulation in automatic synchronization with breathing can be delivered, for example, through an implantable route/electrodes for treating Parkinson's disease, essential tremor, dystonia, epilepsy, obsessive-compulsive disorder, chronic pain, cluster headache, or other diseases; and
Transcranial/transcutaneous magnetic stimulation in automatic synchronization with breathing can be delivered, for example, through electromagnetic induction in which a changing magnetic field is used to cause electric current at a specific area of the brain/organ; for example, this method can be used to treat a variety of disease states, particularly in the fields of FGIDs, neurology and mental health.

In some suitable embodiments, the dose and/or parameters of the electrical stimulations may be titrated while ensuring targeted subtle changes in parasympathetic nerve activity, heart rate, blood and/or other monitored physiological parameters. In practice the waveform and/or other electrical parameters of the stimulations may be selected and/or vary according to the type of therapy and/or treatment being provided. The waveform of the electrical stimulations can be direct current, alternating current, and/or pulsed current. For example, for nerve and/or acupuncture point stimulations, such as vagal nerve, sacral nerve, spinal cord, or acustimulations, the electrical stimulations may be delivered, without limitation, at or with one or more of the following parameters: a frequency in a range of between about 0.5 Hz to about 300 Hz, inclusive, a pulse width in a range of between about 0.1 milliseconds (ms) to about 1.0 ms, inclusive, and a current amplitude in a range of between about 0.1 mA to about 10 mA, inclusive, wherein each pulse train is applied for a duration in a range of between about 1 second to about 120 seconds, inclusive, in a range between about every 10 seconds to about every 300 seconds, inclusive, for up to about 180 minutes. In another example, for organ stimulation, such as gastric electrical stimulation, the frequency may be in a range of between about 3 cycles to about 20 cycle per minute, inclusive, the pulse width may be in a range of between about 0.1 ms to about 600 ms, inclusive, and the current amplitude may be in a range of between about 0.2 mA to about 10 mA, inclusive, wherein the stimulation is applied for up to about 60 minutes. In still other examples, such as for deep brain stimulation, a relatively short pulse width (for example, in a range of between about 10 microseconds (μs) to about 300 μs, inclusive) electrical stimulation may be employed with a somewhat low voltage amplitude (for example, up to about 4 V), a relatively short pulse width electrical stimulation may be employed with a relatively high voltage amplitude (for example, in a range of between about 5 V to about 10 V, inclusive), or a relatively long pulse width (for example, in a range of between about 120 μs to about 450 μs, inclusive) electrical stimulation may be employed with a relatively low voltage amplitude (for example, in a range of between about 0.1 V to about 2 V, inclusive).

In some suitable embodiments, the treatment of functional dyspepsia (FD) may be achieved with the medical treatment device and/or apparatus shown in FIG. 1 and/or the method shown in FIG. 4. FD patients may present with common stomach symptoms without an organic cause. FD can affect a significant portion of the population and can lead to diminished quality of life and a high annual cost for managing the disease. Electrical stimulation in synchronization with breathing can be superior to similar treatments without breathing synchronization, for example, to improve key FD pathophysiologies, including amelioration of impaired gastric accommodation in response to a meal, normalization of gastric dysrhythmias, improvement of visceral hypersensitivity, and enhancement of vagal activity. However, manual breathing synchronization, for example, in which a patient first needs to feel the start of stimulation and manually adjust their respiration to follow the stimulation can have some drawbacks and/or limitations. For example, such manual synchronization can generate considerable delays and/or errors in the synchronization and result in patients' poor compliance with chronic therapy, significantly compromising the effectiveness of this method. In some embodiments, to solve this issue and enhance the effectiveness of electrical stimulation in FD treatment, a novel non-invasive neuromodulation method disclosed herein automatically detects the user's respiration wave and synchronizes electrical stimulation to the user's breathing to deliver those electrical stimulations, for example, at acupuncture points via surface skin electrodes. With the automated synchronization employed by the device and/or apparatus disclosed herein, the patients can inhale and exhale with an uninterrupted and normal respiration pace while receiving the electrical stimulation treatment synchronized therewith, largely simplifying the treatment procedures, improving patients' compliance, and enhancing the effectiveness of the method. The integrative effects of the synchronized electrical stimulations on gastric motility and visceral hyper-sensitivity are based on the activation of vagal pathways. For example, these vagal pathways may include the lateral sural cutaneous branch of the sacral nerve for the ST36 acupoint and/or the median nerve between the tendons of the palmaris longus and flexor carpi radialis muscles for the PC6 acupoint, which jointly induce the beneficial vagally-mediated effects on gastric motility and visceral hypersensitivity. In some embodiments, targeted end organs and related function modulation is as follows: autonomic nerve system, for example, to produce vagal activation; stomach, for example, to improve impaired gastric accommodation and gastric dysrhythmia, such as induced by an Ensure challenge; and visceral hypersensitivity (afferent nerve sensitization), for example, to improve visceral hypersensitivity (FD symptoms), such as provoked by an Ensure challenge. In Some suitable embodiments, transcutaneous electrodes may have a relatively small diameter, for example, about 10 mm, to provide a focal stimulation, for example, at the ST36 and/or PC6 acupoints. In some suitable embodiments, a respiration sensing system (for example, including one or more sensors 10 such as the type disclosed herein) can be employed to measure a user's chest expansions/contractions for respiration monitoring without skin contact. The respiration sensing system may be suitably integrated in a harness or the like that can be readily donned and/or doffed by a user for easy at home self-operation, for example, improving patient compliance. Suitably, in some embodiments, the medical device/apparatus and/or method(s) proposed herein can improve gastric accommodation, normalized gastric dysrhythmia, and ameliorated visceral hypersensitivity, with these improvements mediated by enhanced vagal activity. In some embodiments, for example for FD-specific treatment using acupuncture point ST36 and/or PC6, the electrical stimulation parameters may be as follows: about 0.3 ms to about 0.6 ms, inclusive, pulse width; about 1 Hz to about 100 Hz, inclusive, frequency; about 1 mA to about 10 mA, inclusive, current amplitude; and a cycle of about 1 to 5 seconds on and about 1 to 5 seconds off. In particular embodiments, the on time is shorter than the off time in the cycle, such as about 2 seconds on and 3 seconds off.

In some embodiments, the various controllers, modules, units and/or the like (e.g., such as the system control 30, the stimulation identifier 32, the stimulation controller/driver 34, the feedback module 40, etc.) may be implemented via hardware, software, firmware or a combination thereof. In particular, one or more controllers may be embodied by processors, electrical circuits, computers and/or other electronic data processing devices that are configured and/or otherwise provisioned to perform one or more of the tasks, steps, processes, methods and/or functions described herein. For example, a processor, computer, server or other electronic data processing device embodying a controller may be provided, supplied and/or programmed with a suitable listing of code (e.g., such as source code, interpretive code, object code, directly executable code, and so forth) or other like instructions or software or firmware, such that when run and/or executed by the computer or other electronic data processing device one or more of the tasks, steps, processes, methods and/or functions described herein are completed or otherwise performed. Suitably, the listing of code or other like instructions or software or firmware is implemented as and/or recorded, stored, contained or included in and/or on a non-transitory computer and/or machine readable storage medium or media so as to be providable to and/or executable by the computer or other electronic data processing device. For example, suitable storage mediums and/or media can include but are not limited to: floppy disks, flexible disks, hard disks, magnetic tape, or any other magnetic storage medium or media, CD-ROM, DVD, optical disks, or any other optical medium or media, a RAM, a ROM, a PROM, an EPROM, a FLASH-EPROM, or other memory or chip or cartridge, or any other tangible medium or media from which a computer or machine or electronic data processing device can read and use. In essence, as used herein, non-transitory computer-readable and/or machine-readable mediums and/or media comprise all computer-readable and/or machine-readable mediums and/or media except for a transitory, propagating signal.

In general, any one or more of the particular tasks, steps, processes, methods, functions, elements and/or components described herein may be implemented on and/or embodiment in one or more general purpose computers, special purpose computer(s), a programmed microprocessor or microcontroller and peripheral integrated circuit elements, an ASIC or other integrated circuit, a digital signal processor, a hardwired electronic or logic circuit such as a discrete element circuit, a programmable logic device such as a PLD, PLA, FPGA, Graphical card CPU (GPU), or PAL, or the like. In general, any device, capable of implementing a finite state machine that is in turn capable of implementing the respective tasks, steps, processes, methods and/or functions described herein can be used.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A method of providing electrical stimulation to a patient to treat a disorder from which the patient suffers, said method comprising:
automatically detecting respiration of the patient with a sensor; and
repeatedly administering electrical stimulations to a target site of the patient;
wherein repeated administration of said electrical stimulations is automatically synchronized with the respiration of the patient detected by said sensor.

2. The method of claim 1, further comprising:
obtaining patient data during a period in which prior electrical stimulations had been administered;
determining an effectiveness of the prior electrical stimulations administered during the period based on the obtained patient data; and
adjusting a parameter of future administered electrical stimulations in response to the determined effectiveness.

3. The method of claim 2, wherein the parameter is one of a shape of a waveform of the electrical stimulations, an amplitude of a current of the electrical stimulations, an amplitude of a voltage of the electrical stimulations, a frequency of the electrical stimulations, a pulse width of the electrical stimulations and a duration of the electrical stimulations

4. The method of claim 1, wherein the target site is one of a nerve of the patient and an organ of the patient.

5. The method of claim 1, wherein administering the electrical stimulations comprises:
providing electrical signals to an electrode positioned with respect to the target site of the patient such that the electrical stimulations are administered to the target site.

6. The method of claim 5, wherein the electrode is one of a transcutaneous pad positioned on the patient's skin, a percutaneous needle inserted through the patient's skin, and a lead implanted within the patient.

7. The method of claim 1, wherein a symptom of the disorder is ameliorated by repeated administration of the electrical stimulations automatically synchronized with the respiration of the patient detected by said sensor.

8. The method of claim 1, further comprising:
fitting the sensor to the patient so as to perform said detecting respiration of the patient; and
applying an electrode to the patient, said administering being delivered through said electrode.

9. An apparatus for providing electrical stimulation to a patient to treat a disorder from which the patient suffers, said apparatus comprising:
a sensor which is to be fitted to the patient, said sensor detecting respiration of the patient to which it is fitted; and
an electric power supply, said electric power supply being controlled to provide electrical signals to an electrode such that repeated electrical stimulations are automatically administered to a target site of the patient via said electrode in synchronization with the respiration detected by said sensor.

10. The apparatus of claim 9, further comprising:
a controller in operative communication with said sensor, said controller controlling said electric power supply in response to the respiration detected by said sensor.

11. The apparatus of claim 9, further comprising:
a feedback module comprising at least one processor and at least one memory including computer program code configured to, with the at least one processor, cause the feedback module to at least;
obtain patient data during a period in which prior electrical stimulations had been administered in accordance with a stimulation parameter, said stimulation parameter being one of a shape of a waveform of the electrical stimulations, an amplitude of a current of the electrical stimulations, an amplitude of a voltage of the electrical stimulations, a frequency of the electrical stimulations, a pulse width of the electrical stimulations and a duration of the electrical stimulations;
determine an effectiveness of the prior electrical stimulations administered during the period based on the obtained patient data; and
change the stimulation parameter for future administered electrical stimulations in response to the determined effectiveness.

12. The apparatus of claim 9, wherein the target site is one of a nerve of the patient and an organ of the patient.

13. The apparatus of claim 9, wherein the electrode is one of a transcutaneous pad positioned on the patient's skin, a percutaneous needle inserted through the patient's skin, and a lead implanted within the patient.

14. The apparatus of claim 9, wherein the disorder treated is a functional gastrointestinal disorder.

15. The apparatus of claim 9, wherein a symptom of the disorder is ameliorated by repeated automatic administration of the electrical stimulations synchronized with the detected respiration.

16. The apparatus of claim 9, wherein the sensor is responsive to at least one of expansion and contraction of an anatomy of the patient accompanying respiration by the patient.

17. A medical treatment device that provides electrical stimulation to a user, said device comprising:
a sensor that monitors a respiration cycle of the user;
an electrode that administers electrical stimulations to a target site of the user, wherein said target site is one of a nerve of the user and an organ of the user; and
an electric power supply electrically coupled to the electrode, said electric power supply being controlled to provide electrical signals to the electrode such that repeated electrical stimulations are administered to the target site of the user via said electrode in automatic synchronization with the respiration cycle of the user as monitored by said sensor.

18. The device of claim 17, wherein the electrode comprises one of a transcutaneous pad positioned on a skin of the user, a percutaneous needle inserted through the skin of the user, and a lead implanted within the user.

19. The device of claim 17, wherein a symptom of a disorder suffered by the user is alleviated by repeated administration of the electrical stimulations automatically synchronized with the respiration cycle of the user as monitored by the sensor.

20. The device of claim 17, further comprising:
a controller comprising at least one processor and at least one memory including computer program code configured to, with the at least one processor, cause the controller to at least:
identify one or more points-of-interest within the respiration cycle of the user as monitored by the sensor; and
direct the electric power supplied to provide electrical signals to the electrode at one or more determined times relative to the one or more identified points-of-interest.
